(19) United States
(12) Patent Application Publication
(10) Pub. No.: US 2007/0135638 A1
(43) Pub. Date: Jun. 14, 2007

(54) Process for the manufacture of lysergic acid

(75) Inventors: Ladislav Cvak, Opava (CZ); Vlastislav Mojczek, Hradec Nad Moravici (CZ)

Correspondence Address: LERNER, DAVID, LITTENBERG,;KRUMHOLZ & MENTLIK, 600 SOUTH AVENUE WEST, WESTFIELD, NJ, 07090, US

(73) Assignee: IVAX Pharmaceuticals s.r.o., Opava 9 (CZ)

(21) Appl. No.: 10/589,768

(22) PCT Filed: Feb. 17, 2005
(86) PCT No.: PCT/US05/04933

§ 371 Date: Aug. 17, 2006

Related U.S. Application Data
(60) Provisional application No. 60/546,559, filed on Feb. 20, 2004.

(51) Int. Cl. C07D 457/04

(52) U.S. Cl. 546/69

(57) ABSTRACT

Lysergic acid is formed in high yields and high quality by isomerizing paspalic acid in a phase separated mixture formed by paspalic acid and a concentrated aqueous metal hydroxide solution.

### FIELD OF THE INVENTION

The present invention relates to a process for the manufacture and purification of lysergic acid by isomerizing paspalic acid in high yield.

### BACKGROUND OF THE INVENTION

Lysergic acid is a basic structural part of natural ergot alkaloids. It is manufactured in large scale as an intermediate for the synthesis of several semisynthetic ergot alkaloids, which have found uses as drugs, e.g., ergometrine, methylergometrine, methysergide and nicergoline.

Paspalic acid is also an ergot alkaloid, which is readily available by fermentation. The conversion of paspalic acid to lysergic acid is known by various methods. Current conversion methods, however, result in the formation of unwanted impurities such as the epimer, isolysergic acid, and do not suggest a simple method of removing the isolysergic acid impurity.

Lysergic acid, paspalic acid, and isolysergic acid are natural chiral compounds with the R configuration on the chiral center in position 5 of their skeleton. As used herein, lysergic acid, isolysergic acid, and paspalic acid mean d-lysergic acid, d-isolysergic acid, and d-paspalic acid. Their structures are depicted below.

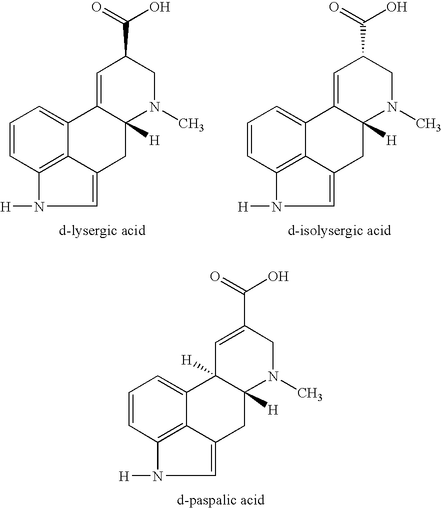

Lysergic acid is typically manufactured by hydrolyzing natural ergot alkaloids like ergotamine or ergotoxine isolated from ergot. Another typical preparation involves the partial synthesis from natural precursors which are available by fermentation, such as by the hydrolysis of lysergic acid hydroxy-ethylamide or the isomerization of paspalic acid.

Paspalic acid and its isomerization to lysergic acid was first described by Kobel (Helv. Chim. Acta 47:1052 (1964)). The isomerization was accomplished by boiling paspalic acid in diluted aqueous sodium hydroxide. When the isomerization of paspalic acid was carried out by this procedure, however, the conversion to lysergic acid was unsatisfactory (more than 5% of paspalic acid remained in the reaction mixture) and the high temperature used caused significant decomposition of the product. Similar results were obtained with other procedures described by Kobel et al. (Helv. Chim. Acta 64:478 (1981) and JP 70013302), both using isomerization in boiling aqueous potassium hydroxide. Moreover, the reaction mixture contained a significant amount of isolysergic acid (more than 25%) which decreased both the yield and the quality of the resulting lysergic acid (the isolated lysergic acid contained about 5% of isolysergic acid). Therefore further purification of the product was necessary. Another drawback was the low concentration of paspalic acid in the reaction mixture, which in turn required a large volume reactor.

Recently a new process of preparing lysergic acid was disclosed, which described the use of aqueous tetraalkylammonium hydroxides to isomerize paspalic acid. (U.S. Pat. No. 6,242,603). The reported yield of isolated lysergic acid containing about 3% of isolysergic acid was about 80%. The content of paspalic acid in the product was not disclosed. This reference also described the preparation of lysergic acid by the above-mentioned process including isomerization in boiling diluted sodium or potassium hydroxide. These comparative examples describe the formation of lysergic acid in less than 60% yields.

Thus, it is desirable to discover a high yielding process for preparing lysergic acid that provides high purity as well.

### SUMMARY OF THE DISCLOSURE

The present invention provides a novel method of preparing lysergic acid from paspalic acid, wherein both high yields and high purity are obtained.

The present invention also provides a novel method of separating isolysergic acid from lysergic acid.

These and other advantages, which will become apparent during the following detailed description, have been obtained by the inventors' discovery that lysergic acid can be formed from paspalic acid and an aqueous solution of a metal hydroxide in a phase separated mixture. In addition, the inventors have also discovered that isolysergic acid can be removed from lysergic acid via a methanol wash. Additional advantages of the present invention will become readily apparent to those skilled in this art from the following detailed description.

### DETAILED DESCRIPTION OF THE DISCLOSURE

In an embodiment, the present invention provides a novel process for the manufacture of lysergic acid, comprising isomerizing paspalic acid in a phase separated mixture, comprising: paspalic acid and an aqueous solution of a metal hydroxide. The amount of the paspalic acid and metal hydroxide is in sufficient quantity to cause a phase separated reaction mixture. The process can advantageously be carried out within a few hours and at relatively low temperature. Isolation of essentially pure lysergic acid can be achieved in yields of greater than 70% and containing less than about 1 wt % of paspalic acid and less than about 1 wt % of isolysergic acid. The process can additionally provide epimerization of unwanted isolysergic acid to the desired lysergic acid. Both the yield and quality of the isolated lysergic acid were substantially better than that known to the art, e.g. see in U.S. Pat. No. 6,242,603 B1.

Sodium hydroxide and potassium hydroxide are both preferred metal hydroxides. Other metal hydroxides (e.g., lithium, rubidium, cesium, magnesium, calcium, strontium, and barium) are expected to be useful in the present invention and are considered part of the present invention.

In order to form a phase separated system, it is preferred that at least about 5, 6, 7, 8, 9, or 10 wt % paspalic acid is present, with about 5 wt % being more preferred. It is also preferred that about a 12, 13, 14, 15, 16, 17, 18, 19, or 20 wt % aqueous solution of metal hydroxide (e.g., sodium and potassium hydroxide) solution is used, with about 12 wt % being more preferred. Weight percentage is determined by divided the weight of the given component by the total weight of the reference. For example, at least a 12% aqueous sodium or potassium hydroxide solution would require at least 12 g of sodium or potassium hydroxide per 100 g of aqueous solution. Another way of saying this is that there would be 12 g of sodium or potassium hydroxide and 88 g of water. Without being bound by any theory, it is believed that the phase separated reaction mixture provides an excellent medium for isomerizing of paspalic acid.

Paspalic acid can be converted to lysergic acid in the above-noted phase separated medium under relatively mild conditions, e.g., after about 4 hours of mixing at a temperature of about 50° C. Under these reaction conditions, the conversion of paspalic acid is preferably greater than 90, 91, 92, 93, 94, 95, 96, 97, 98, or 99%, more preferably greater than 98%. Thus, it is preferably that the reaction mixture after isomerization contains less than about 2.0% of paspalic acid, as determined by HPLC. It is also noted that is it not uncommon for the isomerized paspalic acid to contain a certain amount of isolysergic acid (e.g., about 18% as determined by HPLC).

In general, the present invention is practiced by combining paspalic acid, water and a metal hydroxide. Because of convenience, the paspalic acid is preferably added to the aqueous solution of the metal hydroxide. The ingredients are combined and, optionally, agitated for a time sufficient to achieve high conversion to lysergic acid (e.g., about 1, 2, 3, 4, 5, 6, 7, 8 or more hours). The temperature of the reaction is preferably from about 20, 25, 30, 35, 40, 45, 50, 55, 60, 65, 70, 75, 80, 85, 90, 95, to 100° C., with about 40, 41, 42, 43, 44, 45, 46, 47, 48, 49, 50, 51, 52, 53, 54, 55, 56, 57, 58, 59, 60° C. being more preferred, and 50° C. being even more preferred.

After formation, lysergic acid is preferably separated from the reaction mixture. In another embodiment, lysergic acid is separated from the reaction mixture by acidification to form a crystalline salt of lysergic acid. The acidification preferable lowers the pH of the reaction mixture to 4.0, 3.9, 3.8, 3.7, 3.6, 3.5, or below. Preferably, the acidification is achieved with sulphuric. Sulfuric acid acidification forms the lysergic acid sulfate salt, which precipitates from the reaction mixture. The precipitate is preferably separated from the reaction mixture by methods known to those of ordinary skill in the art (e.g., filtration). Experiments have shown that the crude lysergic acid salt contains roughly the same proportion of lysergic acid and isolysergic acid formed during the isomerizing of paspalic acid. Thus, it is desirable further isolate the formed lysergic acid from its epimer.

In another embodiment, the present invention provides a method of extracting the crude lysergic acid salt with a mixture of an alcohol (e.g., methanol, ethanol, and isopropanol) and aqueous ammonia to regenerate lysergic acid from its salt. Methanol is the preferred alcohol. Preferably, the mixture of alcohol and aqueous ammonia is 95:5 (v/v). After extraction, it is preferable to reduce the volume of the extraction solution. Preferably, this reduction removes nearly all of the ammonia present. It is also preferable to reduce the volume of the alcohol (e.g., methanol) to make it easier to crystallize the lysergic acid.

In another embodiment, the extracted lysergic acid is crystallized. This crystallization can be advantageously aided by the addition of water. The crystallized lysergic acid is then preferably separated leaving behind a mother liquor. This mother liquor can be used in subsequent iterations of the isomerization process. It has been found that the obtained crystalline lysergic acid will still contain some isolysergic acid (e.g., less than about 10%).

In another embodiment, the content of isolysergic acid can be further decreased by washing the crystalline lysergic acid with methanol. It is surprisingly believed that isolysergic acid is more soluble in methanol that lysergic acid. Thus, washing the desired product with methanol provides a convenient way to further purify lysergic acid. It can also be preferable to wash the crystalline material with water prior to washing with methanol. Washing with methanol leaves a methanol wash. This wash can be used in subsequent iterations of the isomerization process. It is preferable to wash the crystalline lysergic acid with methanol to reduce the amount of isolysergic acid impurity to less than about 3 wt %. It is even more preferable to wash the lysergic acid until it contains less than 1% of isolysergic acid.

The mother liquor from the filtration of crystalline lysergic acid and the methanol wash contain mainly isolysergic acid. In another embodiment, these solutions are recycled back to the process of isomerization, where the isolysergic acid is epimerized to the desired lysergic acid at the same time the paspalic acid is isomerized, thereby increasing the overall yield of a multi-batch or continuous process. This recycling is preferably achieved by combing the mother liquor and methanol wash with a second portion of paspalic acid, water, and metal hydroxide (preferably sodium hydroxide or potassium hydroxide). In order to form a phase separated system during the second or more iteration, it is preferred that at least about 5, 6, 7, 8, 9, or 10 wt % paspalic acid is present, with at least about 5 wt % being more preferred and at least about 7 wt % being even more preferred. It is also preferred that about a 12, 13, 14, 15, 16, 17, 18, 19, or 20 wt % aqueous solution of metal hydroxide (e.g., sodium and potassium hydroxide) solution is used, with at least about 12 wt % being more preferred and at least about 15 wt % being even more preferred. The temperature of the second and later isomerizations is preferably the same as described for the first isomerization. Each of the above-noted processes (e.g., acidifying, separating, extracting, reducing, crystallizing, separating, and washing) are performed in the same manner described above.

The recycling of the mother liquors can be repeated 1, 2, 3, 4, 5, 6, 7, 8, 9, or even 10 times and still yield high quality lysergic acid. Each time the mother liquor and methanol wash from the preceding isomerization is combined with a new portion of paspalic acid, water, and metal hydroxide. Preferably, the total yield of a multi-batch process, including the recycling of the mother liquors containing isolysergic acid, is about 70, 75, 80, 85, 90, to 95%, more preferably about 90%. The quality of obtained lysergic acid is very high. Preferably, the average content of paspalic acid is below about 5, 4.5, 4, 3.5, 3, 2.5, 2, 1.5, 1, to 0.5%, more preferably below about 1%. Preferably, the average content of isolysergic acid is below about 5, 4.5, 4, 3.5, 3, 2.5, 2, 1.5, 1, to 0.5%, more preferably below about 1%.

Other features of the invention will become apparent in the course of the following descriptions of exemplary embodiments that are given for illustration of the invention and are not intended to be limiting thereof.

### EXAMPLES

### Example 1

Preparation of Lysergic Acid without Mother Liquor Recycling

Paspalic acid (100.0 g)(titration assay 98.5%) was dissolved in 5% aqueous sodium hydroxide (1000 mL) and then sodium hydroxide (150 g)w as added to the solution. A formation of two-phase mixture was observed. The obtained two-phase mixture was then mixed for about 4 hours at about 50° C. under nitrogen. The reaction mixture was diluted with water (1000 mL), cooled to 10° C., and acidified to a pH of about 3.5 with 40% sulfuric acid. A suspension of crystalline lysergic acid sulfate formed and was mixed for about 2 hours at about 5° C. The crystalline lysergic acid sulfate was filtered off and extracted with a mixture of methanol and aqueous ammonia 95:5 (v/v)(3×500 mL), and the joined extracts were evaporated to about 200 grams, diluted with water (200 mL), and left to crystallize at about 5° C. for 24 hours. The crystalline lysergic acid was then separated and washed with water (100 mL) and methanol (3×100 mL). After vacuum drying (24 hours at 60° C. and 30 mbar), lysergic acid was obtained (73.4 g)(titration assay 99.1%, content of paspalic acid 0.5%, content of isolysergic acid 0.8%).

The mother liquors after crystallization of lysergic acid and the methanol solution obtained after washing the crystalline product were evaporated to a volume of about 200 mL and were then used in Example 2.

### Example 2

Preparation of Lysergic Acid with Mother Liquor Recycling

Sodium hydroxide (50 grams) was dissolved in water (800 mL) and 200 mL of the concentrated mother liquors from the Example 1. Paspalic acid (100.0 g)(titration assay 98.5%) and finally sodium hydroxide (150 g) were added to the solution. A two-phase reaction mixture was subsequently formed and was mixed for about 4 hours at about 50° C. under nitrogen. The reaction mixture was diluted with water (1000 mL), cooled to 10° C., and acidified to about pH 3.5 with 40% sulfuric acid. The obtained suspension was mixed for 2 hours at about 5° C., and the crystalline lysergic acid sulfate was filtered off. The lysergic acid sulfate was extracted with a mixture of methanol and aqueous ammonia 95:5 (v/v)(3×500 mL). The joined extracts were evaporated to about 200 grams, diluted with water (200 mL), and allowed to crystallize at about 5° C. for 24 hours. The crystalline lysergic acid was then separated and washed with water (100 mL) and methanol (3×100 mL). After vacuum drying (24 hours at 60° C. and 30 mbar), lysergic acid (90.8 g) was obtained (titration assay 98.7%, content of paspalic acid 0.6%, content of isolysergic acid 0.9%).

The mother liquors after crystallization of lysergic acid and the methanol solution obtained after washing the crystalline product were evaporated to a volume of about 200 mL and were ready to be used in the next batch.

In this disclosure there is described only the preferred embodiments of the invention and but a few examples of its versatility. It is to be understood that the invention is capable of use in various other combinations and environments and is capable of changes or modifications within the scope of the inventive concept as expressed herein.

What is claimed is:

1. A process for the manufacture of lysergic acid, comprising: isomerizing paspalic acid in a phase separated mixture, comprising: paspalic acid and an aqueous solution of a metal hydroxide.

2. The process of claim 1, wherein the metal hydroxide is selected from sodium hydroxide and potassium hydroxide.

3. The process of claim 2, wherein the phase separated mixture, comprises: at least about 5 wt % of paspalic acid.

4. The process of claim 3, wherein the aqueous solution of sodium hydroxide or potassium hydroxide, comprises: at least about 12 wt % of sodium hydroxide or potassium hydroxide dissolved in water.

5. The process of claim 3, wherein the isomerizing is performed at a temperature in the range of about 40-60° C.

6. The process of claim 2, further comprising: acidifying the reaction mixture after isomerization to form a crystalline salt of lysergic acid.

7. The process of claim 6, wherein the reaction mixture is acidified to a pH of about 4 or below.

8. The process of claim 6, wherein sulfuric acid is used to acidify the reaction mixture.

9. The process of claim 6, further comprising: separating the lysergic acid salt.

10. The process of claim 9, further comprising: extracting lysergic acid from the separated lysergic acid salt with a mixture of an alcohol and aqueous ammonia to form a lysergic acid solution.

11. The process of claim 10, wherein the alcohol is methanol.

12. The process of claim 11, further comprising: reducing the volume of the lysergic acid solution.

13. The process of claim 12, further comprising: crystallizing lysergic acid from the reduced lysergic acid solution.

14. The process of claim 13, wherein the crystallizing is aided by the addition of water.

15. The process of claim 13, further comprising: separating the crystalline lysergic acid to provide crystalline lysergic acid and a first mother liquor.

16. The process of claim 15, further comprising: washing the separated crystalline lysergic acid with methanol to provide crystalline lysergic acid and a first methanol wash.

17. The process of claim 16, wherein the methanol wash is continued until the crystalline lysergic acid comprises less than about 1 wt % paspalic acid and less than about 1 wt % isolysergic acid.

18. The process of claim 16, wherein the yield of lysergic acid obtained is at least 70%.

19. The process of claim 16, further comprising: a second isomerizing of paspalic acid in a phase separated system by combining the first mother liquor and the first methanol wash with a second portion of paspalic acid, water, and a metal hydroxide.

20. The process of claim 19, wherein the first mother liquor and first methanol wash are combined and reduced in volume prior to combining with the second portion paspalic acid, water, and a metal hydroxide.

21. The process of claim 19, wherein the metal hydroxide is sodium or potassium hydroxide.

22. A process of removing isolysergic acid from crystalline lysergic acid, comprising: washing crystalline lysergic acid with methanol.

23. The process of claim 22, wherein the crystalline lysergic acid, comprises at least 5 wt % isolysergic acid.

24. The process of claim 22, wherein the methanol wash is continued until the crystalline lysergic acid comprises less than about 3 wt % of isolysergic acid.

25. The process of claim 24, wherein the methanol wash is continued until the crystalline lysergic acid comprises less than about 1 wt % isolysergic acid.
